(19) United States
(12) Patent Application Publication
(10) Pub. No.: US 2008/0113215 A1
(43) Pub. Date: May 15, 2008

(54) Electroluminescent Materials and Devices

(76) Inventors: Poopathy Kathirgamanathan, North Harrow (GB); Alexander Kit Lay, Reading (GB); Muttulingam Kumaraverl, London (GB); Subramaniam Ganeshamurugan, London (GB)

Correspondence Address: David Silverstein;Andover-IP-Law, 44 Park Street, Suite 300, Andover, MA, 01810, US

(21) Appl. No.: 11/792,160

(22) PCT Filed: Dec. 6, 2005
(86) PCT No.: PCT/GB05/04671

§ 371 Date: Jun. 25, 2007

(30) Foreign Application Priority Data
Dec. 6, 2004 (GB) 0426675.5

(51) Int. Cl. C09K 11/06; C07C 15/00

(52) U.S. Cl. 428/690; 428/411.1; 585/26

(57) ABSTRACT

An electroluminescent compound is a diarylamine anthracene compound.

The present invention relates to electroluminescent materials and to electroluminescent devices.

Materials which emit light when an electric current is passed through them are well known and used in a wide range of display applications. Liquid crystal devices and devices which are based on inorganic semiconductor systems are widely used; however these suffer from the disadvantages of high energy consumption, high cost of manufacture, low quantum efficiency and the inability to make flat panel displays.

Organic polymers have been proposed as useful in electroluminescent devices, but it is not possible to obtain pure colours as they are expensive to make and have a relatively low efficiency.

Another compound which has been proposed is aluminium quinolate, but this requires dopants to be used to obtain a range of colours and has a relatively low efficiency.

Patent application WO98/58037 describes a range of lanthanide complexes which can be used in electroluminescent devices which have improved properties and give better results. Patent Applications PCT/GB98/01773, PCT/GB99/03619, PCT/GB99/04030, PCT/GB99/04024, PCT/GB99/04028, PCT/GB00/00268 describe electroluminescent complexes, structures and devices using rare earth chelates.

U.S. Pat. No. 5,128,587 discloses an electroluminescent device which consists of an organometallic complex of rare earth elements of the lanthanide series sandwiched between a transparent electrode of high work function and a second electrode of low work function with a hole conducting layer interposed between the electroluminescent layer and the transparent high work function electrode and an electron conducting layer interposed between the electroluminescent layer and the electron injecting low work function anode. The hole conducting layer and the electron conducting layer are required to improve the working and the efficiency of the device. The hole transporting layer serves to transport holes and to block the electrons, thus preventing electrons from moving into the electrode without recombining with holes. The recombination of carriers therefore mainly takes place in the emitter layer.

We have now invented electroluminescent compounds and devices incorporating them.

According to the invention there is provided electroluminescent compounds of formula

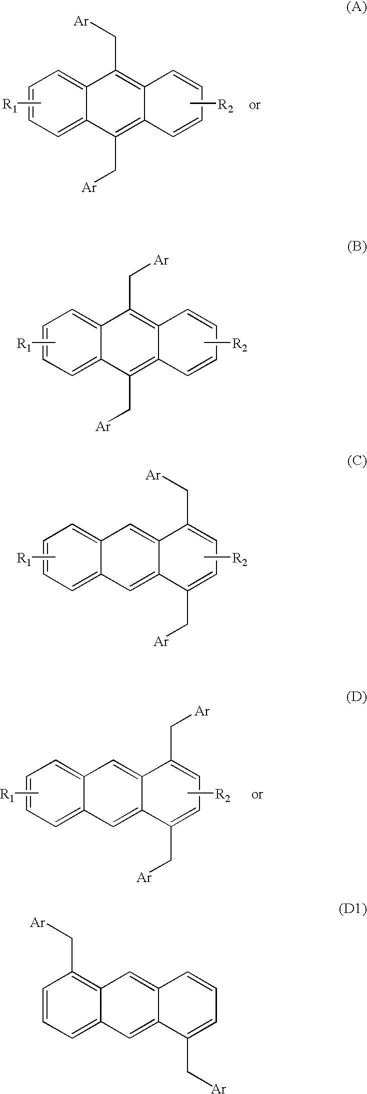

where Ar is an aromatic or a substituted aromatic group or a tertiary alkyl group such as t-butyl and R₁ and R₂ are the same or different and are selected from hydrogen, and substituted and unsubstituted hydrocarbyl groups such as substituted and unsubstituted aliphatic groups, substituted and unsubstituted aromatic, heterocyclic and polycyclic ring structures, fluorocarbons such as trifluoryl methyl groups, halogens such as fluorine or thiophenyl groups; R₁, and R₂ can also form substituted and unsubstituted fused aromatic, heterocyclic and polycyclic ring structures and can be copolymerisable with a monomer e.g. styrene.

Examples of groups Ar are

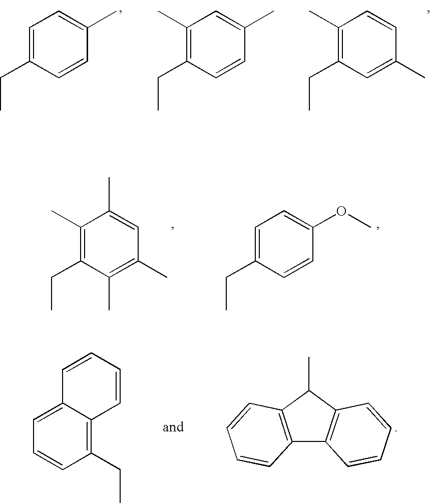

The compounds of the present invention are sterically hindered because of the size of the substituents group on the anthracene ring and any substituents group which cause the substituted anthracene molecule to be sterically hindered can be used.

The compounds of the present invention have a high melting point Tm compared with many other electroluminescent compounds which makes them easier to fabricate an electroluminescent device incorporating them more stable, e.g. above 100° C. with many compounds above 200° C.

The invention also provides an electroluminescent device which comprises (i) a first electrode, (ii) a layer of an electroluminescent compound of formula (A), (B), (C) or (D) above and (iii) a second electrode.

The first electrode can function as the cathode and the second electrode can function as the anode and preferably there is a layer of a hole transporting material between the anode and the layer of the electroluminescent compound.

The hole transporting material can be any of the hole transporting materials used in electroluminescent devices.

The electroluminescent material can be mixed with a host and preferably the host forms a common phase with the electroluminescent material.

Preferred host materials are conjugated aromatic compounds of formula: —

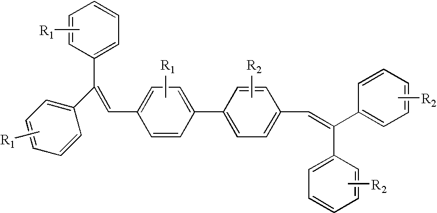

Where R1 and R2 can be hydrogen or substituted or unsubstituted hydrocarbyl groups, such as substituted and unsubstituted aromatic, heterocyclic and polycyclic ring structures,

The hole transporting material can be an amine complex such as poly (vinylcarbazole), N,N′-diphenyl-N,N′-bis(3-methylphenyl)-1,1′-biphenyl-4,4′-diamine (TPD), an unsubstituted or substituted polymer of an amino substituted aromatic compound, a polyaniline, substituted polyanilines, polythiophenes, substituted polythiophenes, polysilanes etc. Examples of polyanilines are polymers of

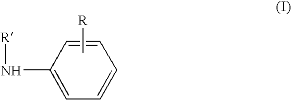

where R is in the ortho—or meta-position and is hydrogen, C1-18 alkyl, C1-6 alkoxy, amino, chloro, bromo, hydroxy or the group

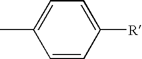

where R is alky or aryl and R′ is hydrogen, C₁₋₆ alkyl or aryl with at least one other monomer of formula (I) above.

Or the hole transporting material can be a polyaniline. Polyanilines which can be used in the present invention have the general formula

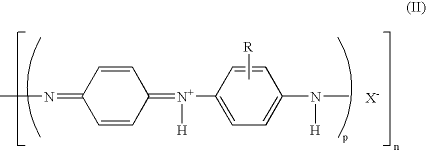

where p is from 1 to 10 and n is from 1 to 20, R is as defined above and X is an anion, preferably selected from Cl, Br, SO₄, BF₄, PF₆, H₂PO₃, H₂PO₄, arylsulphonate, arenedicarboxylate, polystyrenesulphonate, polyacrylate alkysulphonate, vinylsulphonate, vinylbenzene sulphonate, cellulose sulphonate, camphor sulphonates, cellulose sulphate or a perfluorinated polyanion.

Examples of arylsulphonates are p-toluenesulphonate, benzenesulphonate, 9,10-anthraquinone-sulphonate and anthracenesulphonate; an example of an arenedicarboxylate is phthalate and an example of arenecarboxylate is benzoate.

We have found that protonated polymers of the unsubstituted or substituted polymer of an amino substituted aromatic compound such as a polyaniline are difficult to evaporate or cannot be evaporated, however we have surprisingly found that if the unsubstituted or substituted polymer of an amino substituted aromatic compound is deprotonated then it can easily be evaporated i.e. the polymer is evaporable.

Preferably evaporable deprotonated polymers of unsubstituted or a substituted polymer of an amino substituted aromatic compound are used. The de-protonated unsubstituted or substituted polymer of an amino substituted aromatic compound can be formed by deprotonating the polymer by treatment with an alkali such as ammonium hydroxide or an alkali metal hydroxide such as sodium hydroxide or potassium hydroxide.

The degree of protonation can be controlled by forming a protonated polyaniline and de-protonating. Methods of preparing polyanilines are described in the article by A. G. MacDiarmid and A. F. Epstein, Faraday Discussions, Chem. Soc. 88 P37 789.

The conductivity of the polyaniline is dependent on the degree of protonation with the maximum conductivity being when the degree of protonation is between 40 and 60% e.g. about 50%.

Preferably the polymer is substantially fully deprotonated.

A polyaniline can be formed of octamer units, i.e. p is four, e.g.

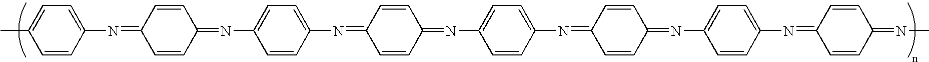

The polyanilines can have conductivities of the order of 1×10⁻¹ Siemen cm⁻¹ or higher.

The aromatic rings can be unsubstituted or substituted e.g. by a C1 to 20 alkyl group such as ethyl.

The polyaniline can be a copolymer of aniline and preferred copolymers are the copolymers of aniline with o-anisidine, m-sulphanilic acid or o-aminophenol, or o-toluidine with o-aminophenol, o-ethylaniline, o-phenylene diamine or with amino anthracenes.

Other polymers of an amino substituted aromatic compound which can be used include substituted or unsubstituted polyaminonapthalenes, polyaminoanthracenes, polyaminophenanthrenes, etc. and polymers of any other condensed polyaromatic compound. Polyaminoanthracenes and methods of making them are disclosed in U.S. Pat. No. 6,153,726. The aromatic rings can be unsubstituted or substituted e.g. by a group R as defined above.

Other hole transporting materials are conjugated polymers and the conjugated polymers which can be used can be any of the conjugated polymers disclosed or referred to in U.S. Pat. No. 5,807,627, PCT/WO90/13148 and PCT/WO92/03490.

The preferred conjugated polymers are poly(p-phenylenevinylene)-PPV and copolymers including PPV. Other preferred polymers are poly(2,5 dialkoxyphenylene vinylene) such as poly(2-methoxy-5-(2-methoxypentyloxy-1,4-phenylene vinylene), poly(2-methoxypentyloxy)-1,4-phenylenevinylene), poly(2-methoxy-5-(2-dodecyloxy-1,4-phenylenevinylene) and other poly(2,5 dialkoxyphenylenevinylenes) with at least one of the alkoxy groups being a long chain solubilising alkoxy group, poly fluorenes and oligofluorenes, polyphenylenes and oligophenylenes, polyanthracenes and oligo anthracenes, ploythiophenes and oligothiophenes.

In PPV the phenylene ring may optionally carry one or more substituents e.g. each independently selected from alkyl, preferably methyl, alkoxy, preferably methoxy or ethoxy.

Any poly(arylenevinylene) including substituted derivatives thereof can be used and the phenylene ring in poly(p-phenylenevinylene) may be replaced by a fused ring system such as anthracene or naphthlyene ring and the number of vinylene groups in each polyphenylenevinylene moiety can be increased e.g. up to 7 or higher.

The conjugated polymers can be made by the methods disclosed in U.S. Pat. No. 5,807,627, PCT/WO90/13148 and PCT/WO92/03490.

The thickness of the hole transporting layer is preferably 20 nm to 200 nm.

The polymers of an amino substituted aromatic compound such as polyanilines referred to above can also be used as buffer layers with or in conjunction with other hole transporting materials.

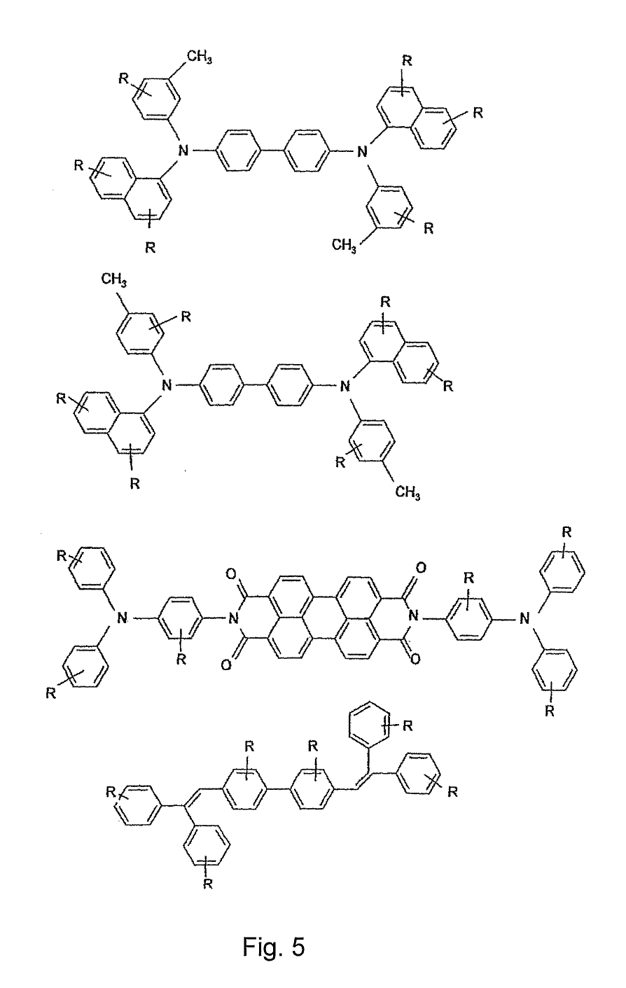

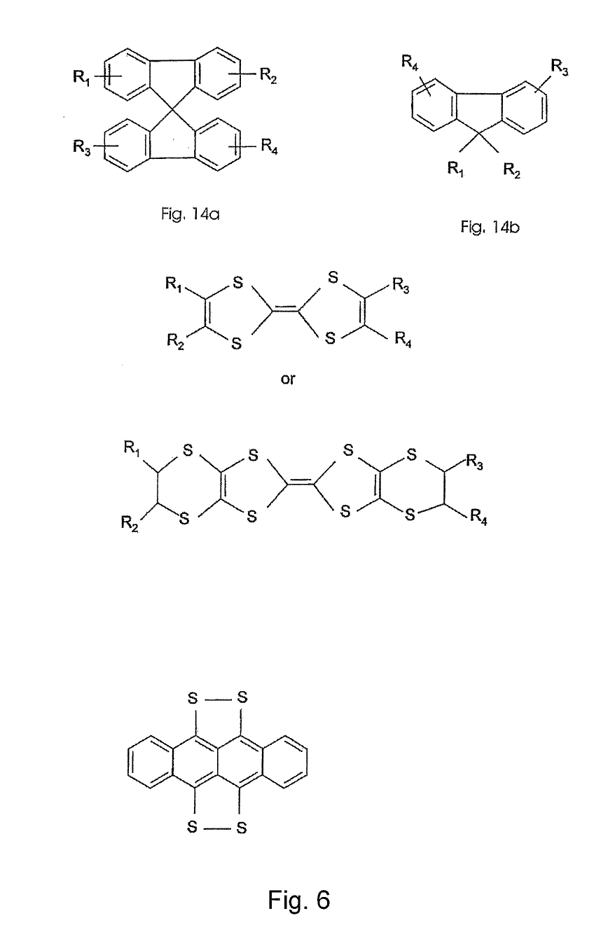

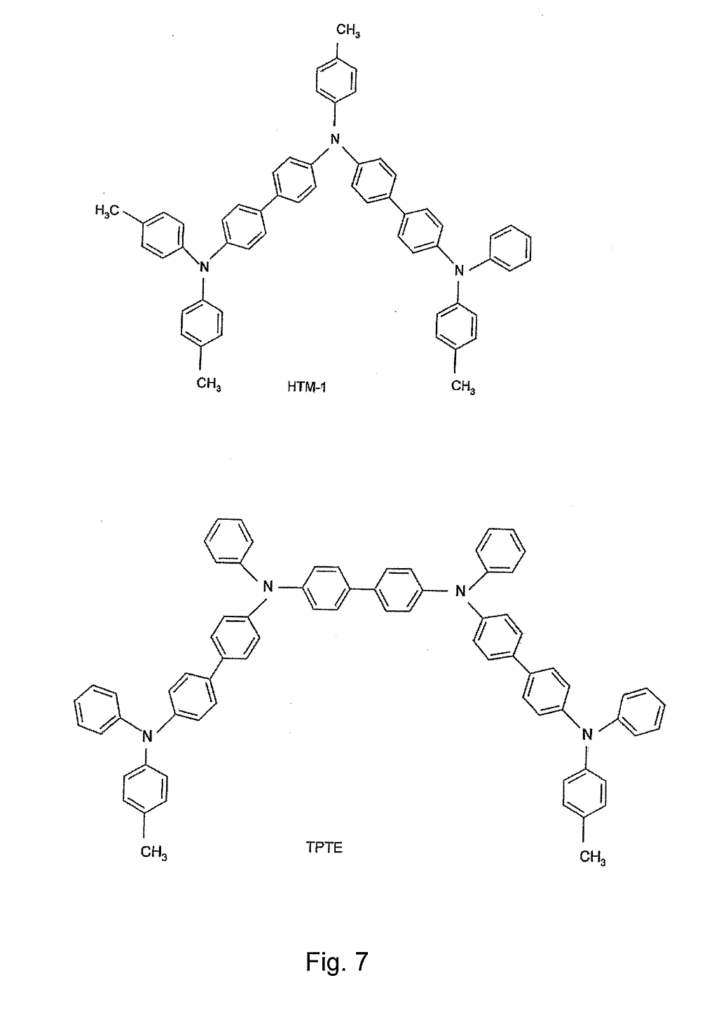

The structural formulae of some other hole transporting materials are shown in FIGS. 5, 6, 7 and 8 of the drawings, where R₁, R₂ and R₃ can be the same or different and are selected from hydrogen, and substituted and unsubstituted hydrocarbyl groups such as substituted and unsubstituted aliphatic groups, substituted and unsubstituted aromatic, heterocyclic and polycyclic ring structures, fluorocarbons such as trifluoryl methyl groups, halogens such as fluorine or thiophenyl groups; R₁, R₂ and R₃ can also form substituted and unsubstituted fused aromatic, heterocyclic and polycyclic ring structures and can be copolymerisable with a monomer e.g. styrene. X is Se, S or O, Y can be hydrogen, substituted or unsubstituted hydrocarbyl groups, such as substituted and unsubstituted aromatic, heterocyclic and polycyclic ring structures, fluorine, fluorocarbons such as trifluoryl methyl groups, halogens such as fluorine or thiophenyl groups or nitrile.

Examples of R₁ and/or R₂ and/or R₃ include aliphatic, aromatic and heterocyclic alkoxy, aryloxy and carboxy groups, substituted and substituted phenyl, fluorophenyl, biphenyl, phenanthrene, anthracene, naphthyl and fluorene groups alkyl groups such as t-butyl, heterocyclic groups such as carbazole.

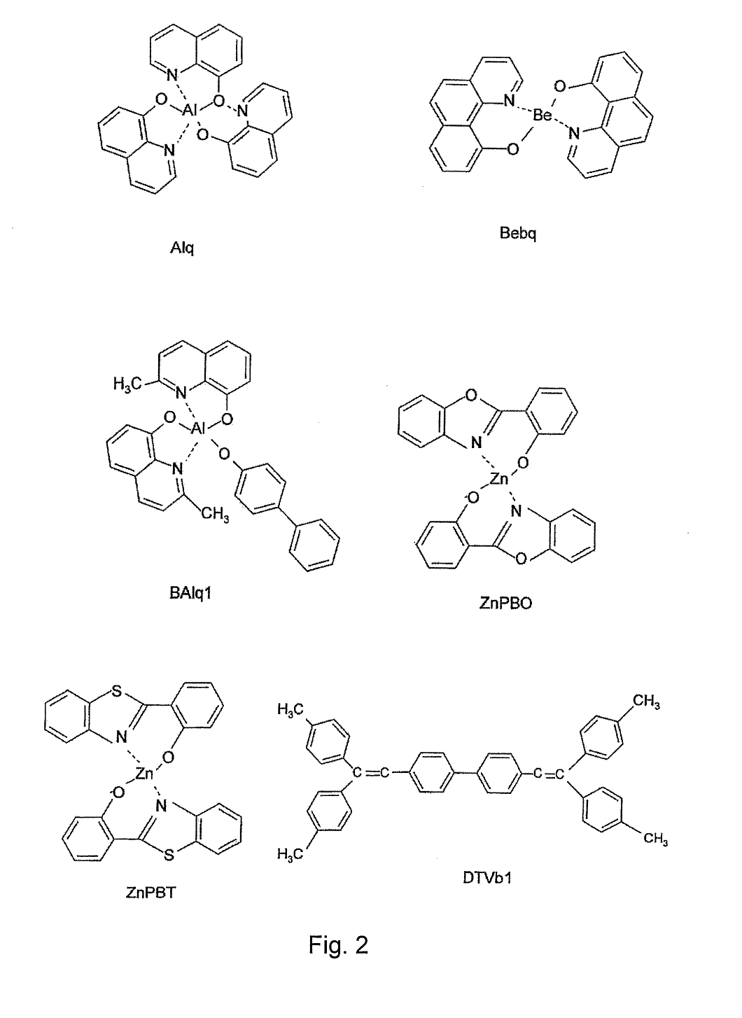

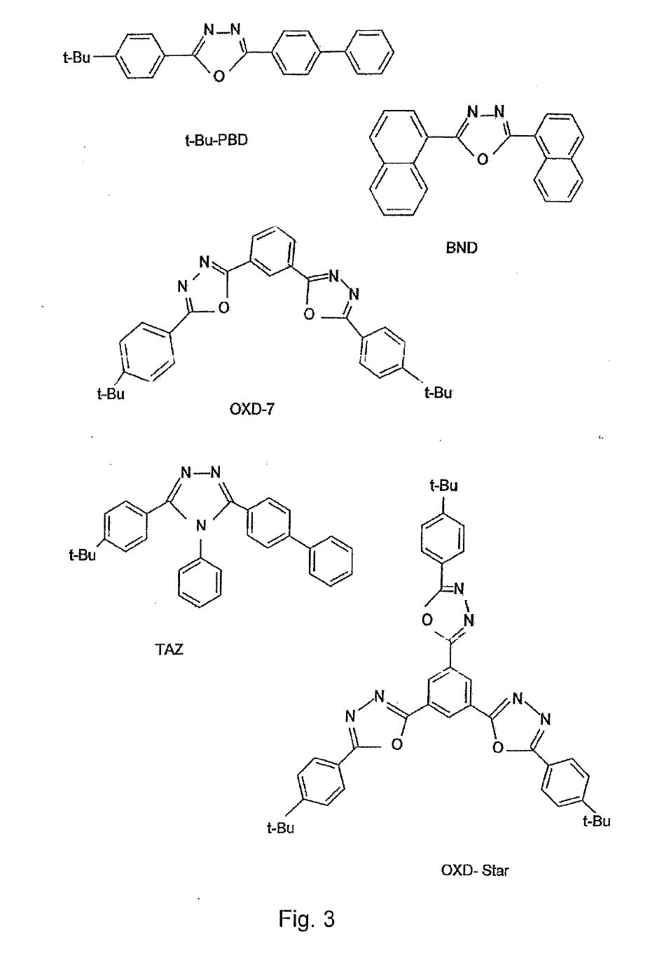

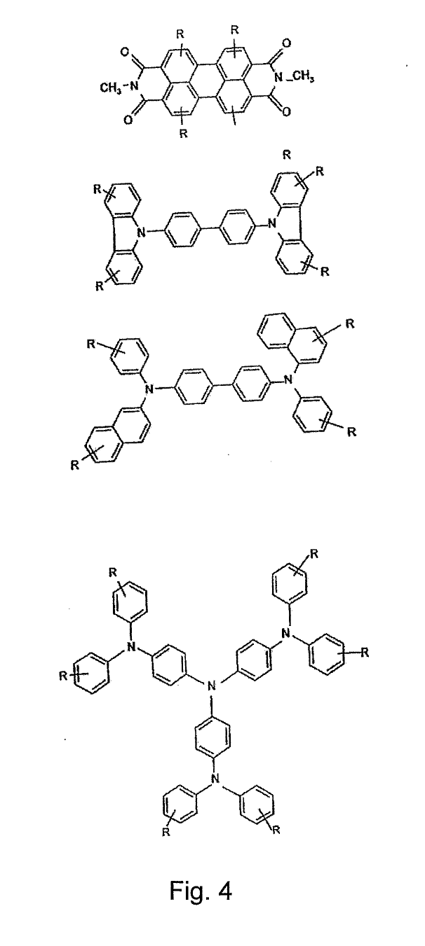

Optionally there is a layer of an electron injecting material between the cathode and the electroluminescent composition layer; the electron injecting material is a material which will transport electrons when an electric current is passed through electron injecting materials and include a metal complex such as a metal quinolate e.g. an aluminium quinolate, lithium quinolate, zirconium quinolate; a compound of formula Mx(DBM)ₙ where Mx is a metal and DBM is dibenzoyl methane and n is the valency of Mx, e.g. Mx is chromium. The electron injecting material can also be a cyano anthracene such as 9,10 dicyano anthracene, cyano substituted aromatic compounds, tetracyanoquinidodimethane, a polystyrene sulphonate or a compound with the structural formulae shown in FIG. 2 or 3 of the drawings in which the phenyl rings can be substituted with substituents R as defined above; or a metal thioxinate of formula

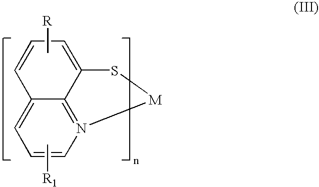

where M is a metal, preferably zinc, cadmium, gallium and indium; n is the valency of M; R and R₁ which can be the same or different are selected from hydrogen, and substituted and unsubstituted hydrocarbyl groups such as substituted and unsubstituted aliphatic groups, substituted and unsubstituted aromatic, heterocyclic and polycyclic ring structures, fluorocarbons such as trifluoryl methyl groups, halogens such as fluorine; thiophenyl groups; cyano group; substituted and unsubstituted hydrocarbyl groups such as substituted and unsubstituted aliphatic groups, substituted and unsubstituted aliphatic groups as described in patent application PCT/GB2005/002579.

The electron injecting material layer should have a thickness so that the holes form the anode and the electrons from the cathode combine in the electroluminescent layer.

### EXAMPLES

### Synthesis for 9,10-Dibenzylanthracene Compounds

This is a general synthesis for these compounds. In each separate case a different benzyl chloride compound is used.

Anthracene (8.0 g, 44.9 mmol), Zinc dust (2.35 g, 35.9 mmol) and the benzyl chloride (94 mmol) were stirred in carbon disuiphide (150 ml) and refluxed for 30 h. The reaction was cooled to room temperature and the solvent was removed by distillation. The residue was extracted into hot toluene (200 ml) and filtered under vacuum to remove excess zinc. On cooling, the toluene solution yielded a light yellow crystalline product which was recrystallised from hot toluene, filtered and dried in a vacuum oven.

### Example 1

For 9,10-Bis(4-methyl-benzyl)-anthracene (E)

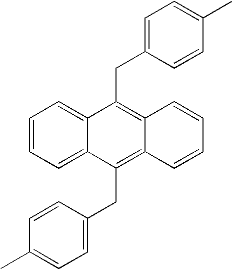

### 4-Methylbenzylchloride was used.

### Example 2

For 9,10-Bis-(2,4-dimethyl-benzyl)-anthracene (F)

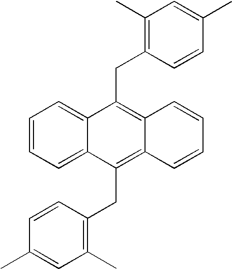

### 2,4-Dimethylbenzyl chloride was used.

### Example 3

For 9,10-Bis-(2,5-dimethyl-benzyl)-anthracene (G)

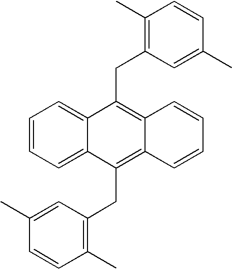

### 2,5-Dimethylbenzyl chloride was used.

### Example 4

For 1,4-Bis-(2,3,5,6-tetramethyl-benzyl)-anthracene (H)

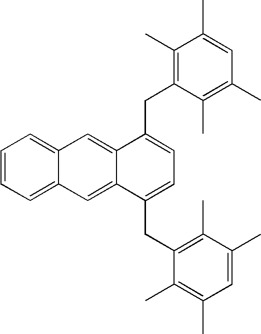

2,3,5,6-tetrameyhylbenzyl chloride was used.

### Example 5

For 9,10-Bis-(4-methoxy-benzyl)-anthracene (J)

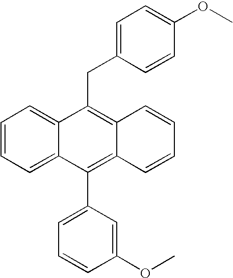

### 4-Methoxybenzyl chloride was used.

### Example 6

For 9,10-Bis-(9H-fluoren-9-yl)-anthracene (L)

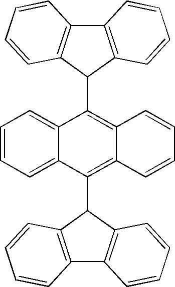

### 9-Bromofluorene was used.

### Example 7

### Preparation of 2,6-Di-tert-butyl-anthracene (N)

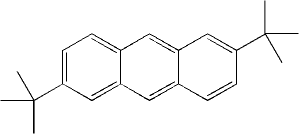

Anthracene (7.13 g. 40 mmol) and tert-Butanol (10.8 g, 120 mmol) were refluxed for 15 h in Trifluoroacetic acid (40 ml). The mixture was cooled and poured into water (250 ml). The solid that formed was filtered under vacuum, washed with water and dried. The solid was recrystallised from hot hexane to yield a colourless crystalline solid. M.p. 249-253° C.

### Example 8

### Preparation of 2,6-Di-tert-butyl-9,10-bis-(2,5-dimethyl-benzyl)-anthracene (O)

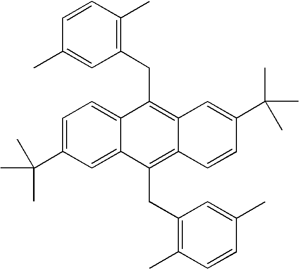

N (Example 7) (3.0 g, 10.3 mmol), Zinc dust (0.54 g, 8.3 mmol) and 2,5-Dimethylbenzyl chloride (3.35 g, 21.7 nmol) were stirred in carbon disulphide (50 ml) and refluxed for 30 h. The reaction was cooled to room temperature and the solvent was removed by distillation. The residue was extracted into hot toluene (50 ml) and filtered under vacuum to remove excess zinc. On cooling, the toluene solution yielded a colourless crystalline product which was recrystallised from hot toluene, filtered and dried in a vacuum oven. M.p. 273-275° C.

### Example 9

### Preparation of 2,6-Di-tert-butyl-9,10-bis-naphthalen-1-ylmethyl-anthracene (S)

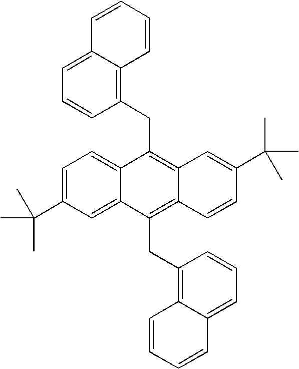

N (Example 7) (2.9 g, 10 mmol), Zinc dust (0.52 g, 8 mmol) and 1-(chloromethyl)naphthalene (3.7 g, 20.9 mmol) were stirred in carbon disulphide (50 ml) and refluxed for 30 h. The reaction was cooled to room temperature and the solvent was removed by distillation. The residue was extracted into hot toluene (50 ml) and filtered under vacuum to remove excess zinc. On cooling. The toluene solution yielded a colourless crystalline product which was recrystallised from hot toluene, filtered and dried in a vacuum oven. M.p. 285° C.

The photoluminescent properties and fluorescence were measured and the results shown in the accompanying table. The colour coordinates were measured on the CIE 1931 Chromacity Diagram and, as can be seen, the compounds emitted a purple blue colour. Compound (M) of the table were made by analogous methods to Example 1.

Photoluminescence was excited using 325 nm line of Liconix 4207 NB, He/Cd laser. The laser power incident at the sample (0.3 mWcm⁻²) was measured by a Liconix 55PM laser power meter. The radiance calibration was carried out using Bentham radiance standard Bentham SRS8, Lamp current 4,000A, calibrated by National Physical laboratories, England.

TABLE

| | | | | | CIE co- |
|---|---|---|---|---|---|
| | | | Fluorescence | | ord |
| | | Fluorescence | Thin Film | | (254 |
| Compound | P.E. Data | Powder | (~100 nm) | DSC | exctn) |
| 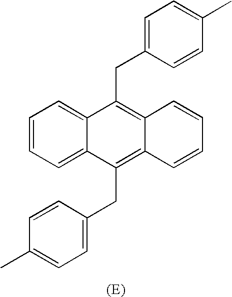 | Eff0.067 cdm⁻²μW⁻¹Peak: ~465 nmFWHM: ~45 nmX: 0.14 y: 0.12Brightnessdrops0.1 cdm⁻²s⁻¹ | Emission max:470.5 nmExcitation max447.7 nm | Emission max:447 nmExcitation max410 nm | Tm:250° C.onset | x: 0.145y: 0.104 |
| 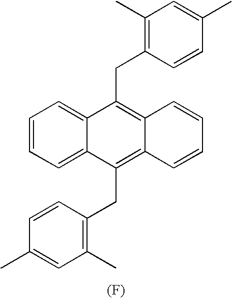 | Eff0.031 cdm⁻²μW⁻¹Peak: ~465 nmFWHM: ~45 nmx: 0.15 y: 0.12 | Emission max:469.2 nmExcitation max441.6 nm | Emission max:446 mExcitation max404 mBroad | Tm:267° C.onset | x: 0.147y: 0.097 |
| 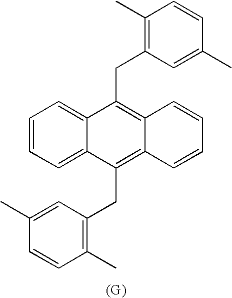 | Eff0.061 cdm⁻²μW⁻¹Peak: ~465 nmFWHM: 45 nmX: 0.14 y: 0.15Brightnessdrops0.1 cdm⁻²s⁻¹ | Emission max:447 nmExcitation max416 nm | Emission max:456 nmExcitation max403 nmBroad Shoulderat 495 nm | Tm:297° C.onset | |
| 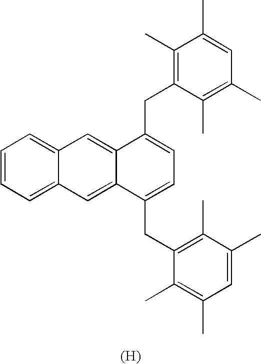 | Disc.Eff0.002 cdm⁻²μW⁻¹Peak: ~443 nmX: 0.16 y: 0.09 | Emission max:418.95 nmExcitation max390.4 nm | Emission max:450 nmExcitation max390 nmBroad Shoulderat 450 nm | Tm:>370° C. | x: 0.16y: 0.07 |
| 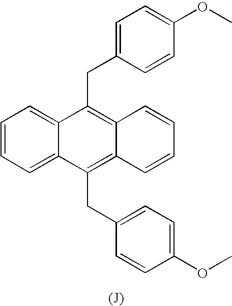 | Disc.Eff0.002 cdm⁻²μW⁻¹ | Emission max:452.4 nmExcitation max398.9 nm | Emission max:453 nmExcitation max406 nmBroaded | Tm:222-227° C. | x: 0.16y: 0.09 |
| 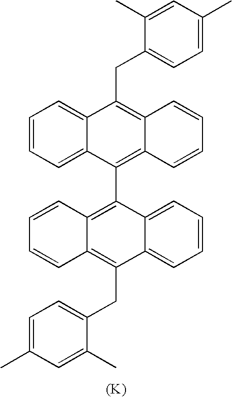 | Eff0.001 cdm⁻²μW⁻¹Drops overtimeX: 0.16 y: 0.14Peak ~450 nm | Emission max:445 nmExcitation max421 nm | | Tg:116° C. | x: 0.16y: 0.07 |
| 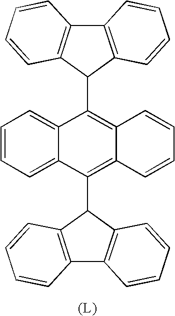 | X: 0.18 Y: 0.37Efficiency0.060 cdm⁻²μW⁻¹ | Emission peakmax 467 nmFWHM~75 nmExcitation max412 nm | Emission max:435 nmExcitation max493 nmNarrowed | Tm:374-378° C. | x: 0.14y: 0.19 |
| 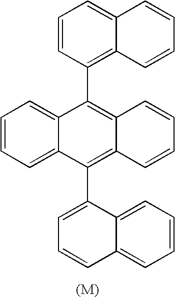 | X: 0.167Y: 0.153Efficiency0.060 cdm⁻²μW⁻¹ | Emission peakmax 442 nmFWHM~15 nmExcitation max392 nm | | Tm:354-361° C. | x: 0.16y: 0.08 |
| 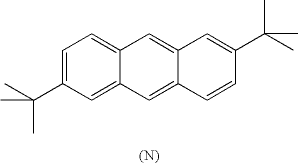 | X: 0.16 Y: 0.08Efficiency0.019 cdm⁻²μW⁻¹ | Emission peakmax 424 nmFWHM~35 nmExcitation max393 nm | | Tm:249-253° C. | x: 0.16y: 0.05 |
| 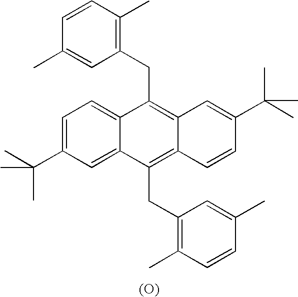 | X: 0.15 Y: 0.08Efficiency0.073 cdm⁻²μW⁻¹ | Emission peakmax 429 nm(442 nmsecondary)FWHM~45 nmExcitation max392 nm | Emission max:427 nmShoulder at 445 nm | Tm:74-277° C. | x: 0.15y: 0.05 |
| 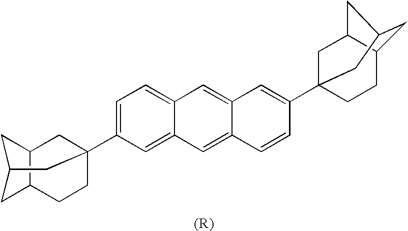 | X: 0.16 Y: 0.05Efficiency0.011 cdm⁻²μW⁻¹ | X: 0.16 Y: 0.03Emission peakmax 412 nm(436 nmsecondary)FWHM~15 nmExcitation max382 nm | Emission max:413 nmExcitation max364 nmSecondary peaksat 394 nm and436 nm | Tm:378-382° C. | x: 0.16y: 0.03 |
| 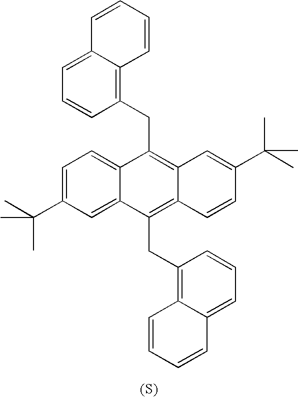 | X: 0.16 Y: 0.1Efficiency0.29 cdm⁻²μW⁻¹ | Emission peakmax 437 nmFWHM~40 nmExcitation max393 nm | Thin FilmEmission peakmax.452 nmFWHM~450 nmExcitation max402 nm | Tm:>285° C. | x: 0.16y: 0.05 |

### Electroluminscent Devices

### Example 10

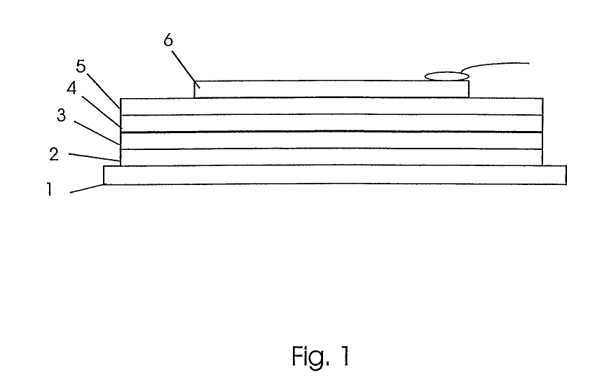

A pre-etched ITO coated glass piece (10×10 cm²) was used. The device was fabricated by sequentially forming on the ITO, by vacuum evaporation, the compositions forming the layers comprising the electroluminescent device. The layers were deposited using a Solciet Machine, ULVAC Ltd. Chigacki, Japan. The active area of each pixel was 3 mm by 3 mm; the device is shown in FIG. 1 and the layers comprised: —

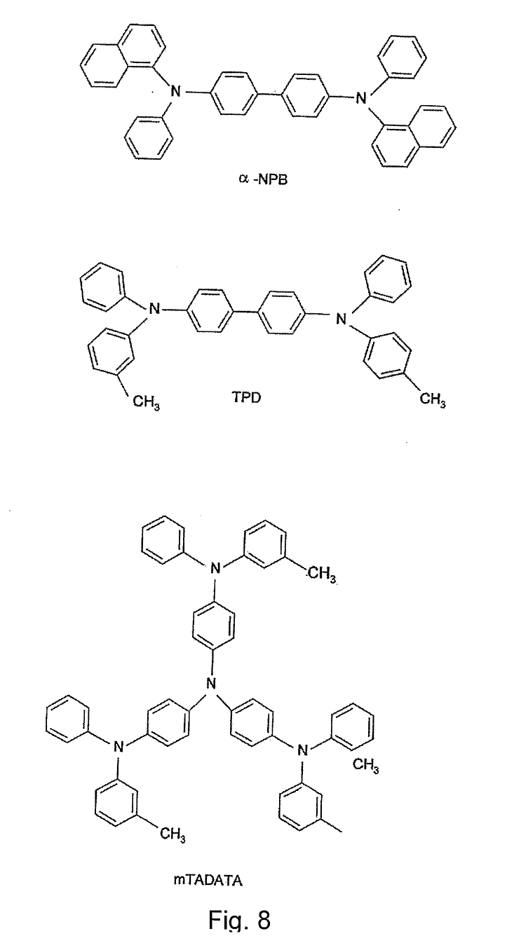

### (1) ITO (100 nm)/(2)CuPc (25 nm)/(3)α-NPB (55 nm)/(4) Compound Q:Compound S (30:3 nm)/(5)Hfq₄ (20 nm)/(6)LiF (0.3 nm)/Al

where ITO is indium tin oxide coated glass, α-NPB is shown in FIG. 8 of the drawings, Hfq₄ is hafnium quinolate, CuPc is copper phthalocyanine and S and Q are as shown below.

The coated electrodes were stored in a vacuum desiccator over a molecular sieve and phosphorous pentoxide until they were loaded into a vacuum coater (Edwards, 10⁻⁶ torr) and aluminium top contacts made. The devices were then kept in a vacuum desiccator until the electroluminescence studies were performed.

The ITO electrode was always connected to the positive terminal. The current vs. voltage studies were carried out on a computer controlled Keithly 2400 source meter.

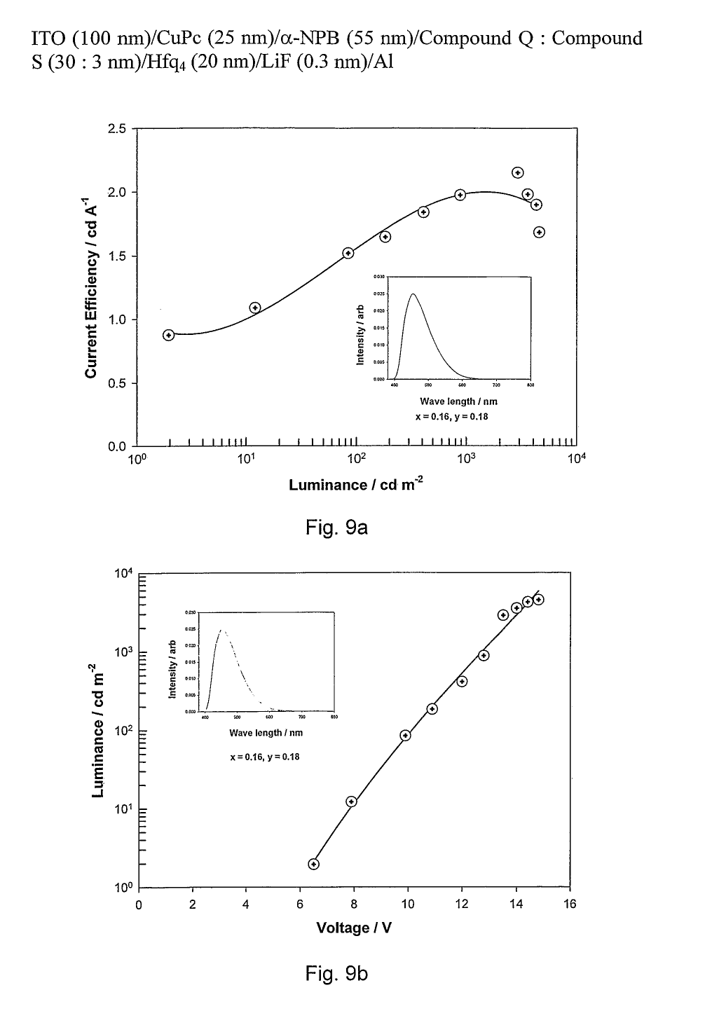

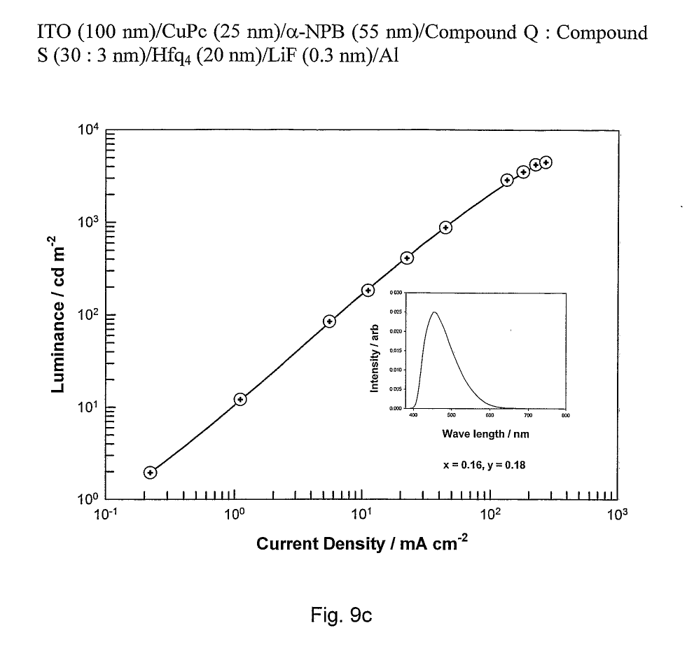

A voltage was applied across the device and the properties measured and the results are shown in FIGS. 9a, 9b and 9c.

### Example 11

A device was formed as in Example 10 with the structure: —

### ITO (100 μm)/Compound X (20 nm)/α-NPB (65 nm)/Compound Q:Compound S (25:1 nm)/Hfq₄ (20 nm)/LiF (0.3 nm)/Al

where X, S and Q are as shown below.

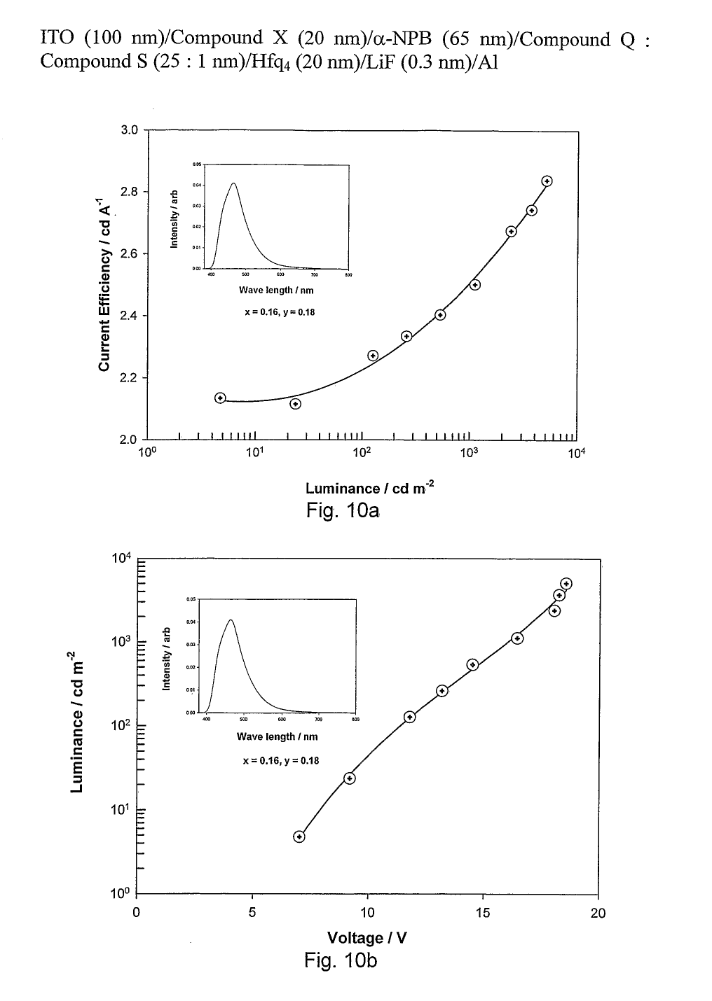

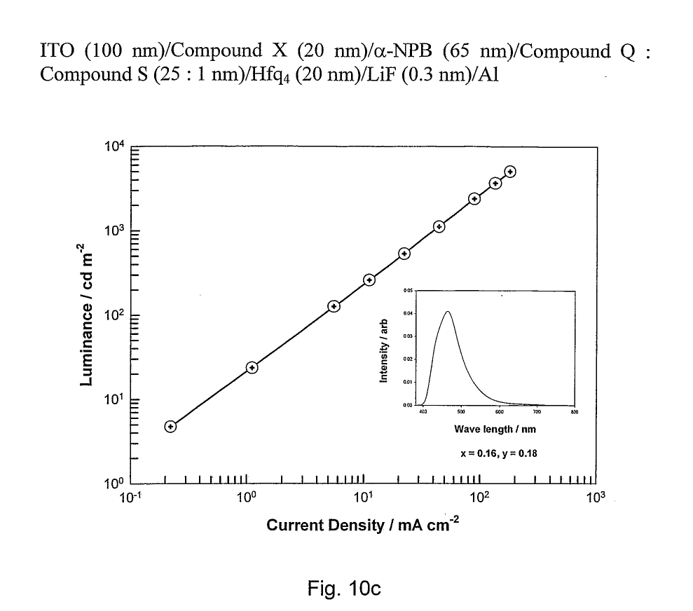

A voltage was applied across the device and the properties measured and the results are shown in FIGS. 10a, 10b and 10c.

### Example 12

A device was formed as in Example 10 with the structure: —

### ITO (100 nm)/ZnTpTP (20 nm)/α-NPB (65 nm)/Compound Q:Compound S (25:1 nm)/Hfq₄ (20 nm)/LiF (0.3 nm)/Al

where ZnTpTp, S and Q are as shown below.

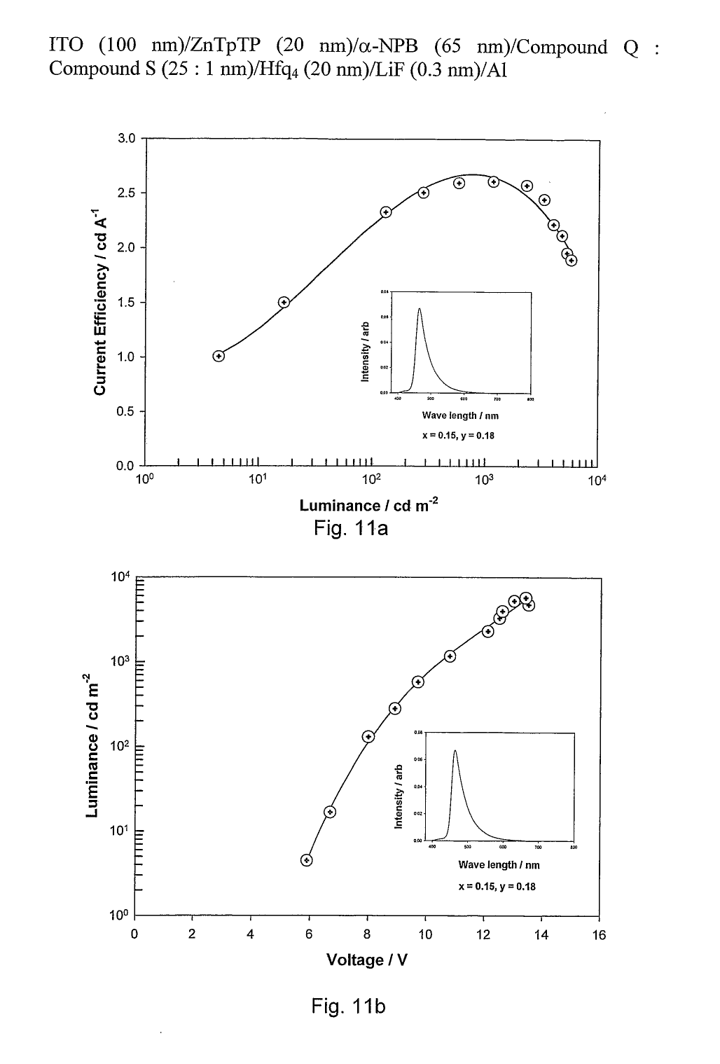

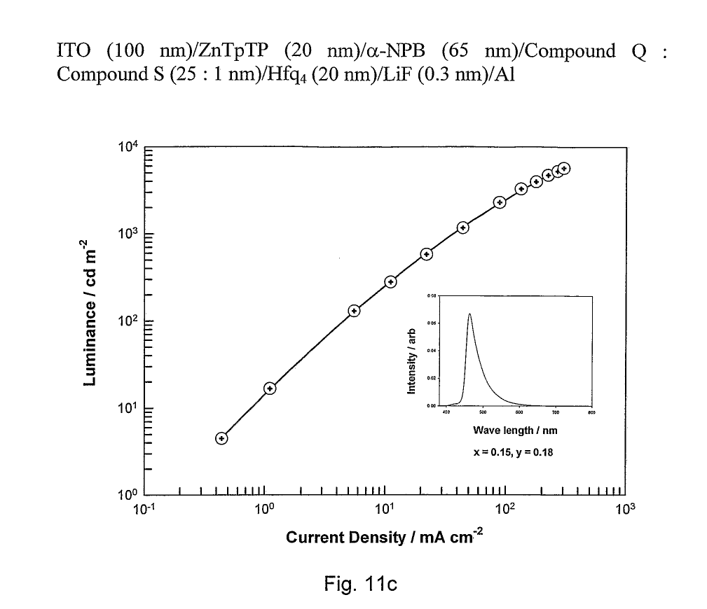

A voltage was applied across the device and the properties measured and the results are shown in FIGS. 11a, 11b and 11c.

### Example 13

A device was formed as in Example 10 with the structure: —

### ITO (150 nm)/CuPc (50 nm)/α-NPB (60 nm)/Compound S:perylene (40:0.34 nm)/Zrq₄ (20 nm)/LiF (0.5 nm)/Al

where S is as shown below.

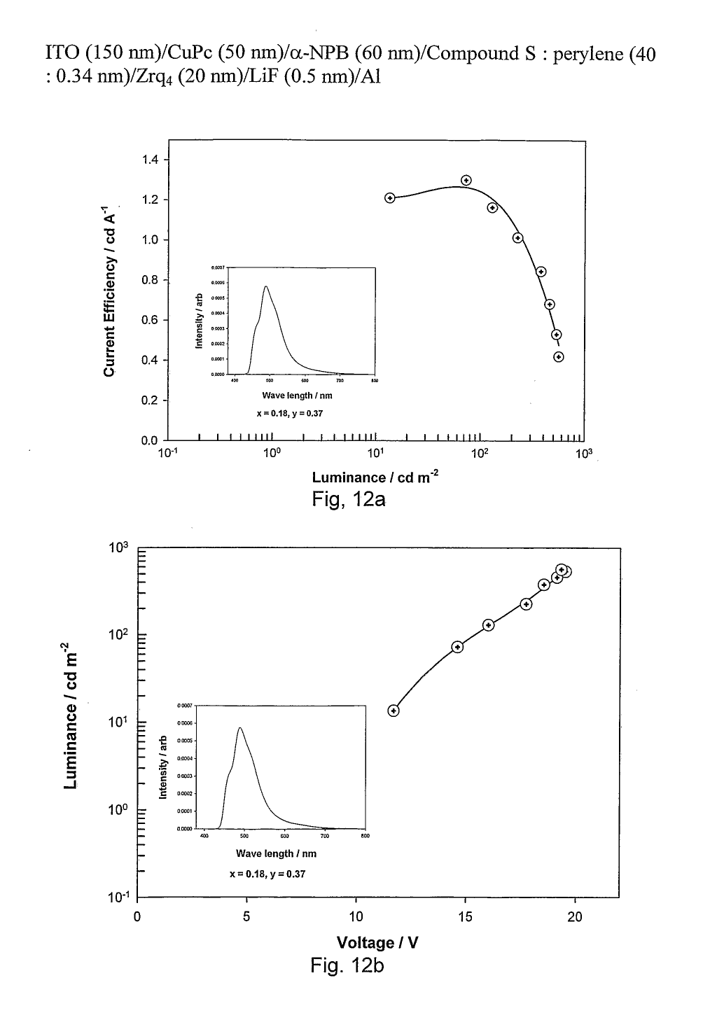

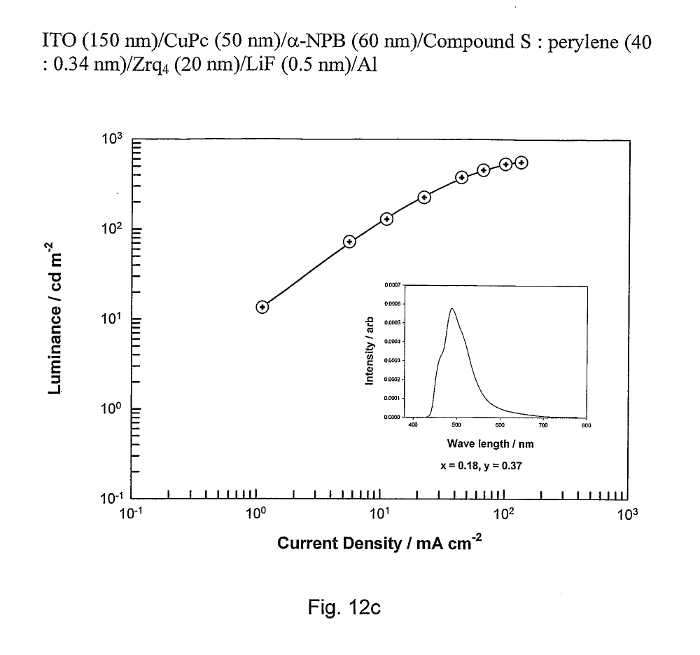

A voltage was applied across the device and the properties measured and the results are shown in FIGS. 12a, 12b and 12c.

### Example 14

A device was formed as in Example 10 with the structure: —

### ITO (150 nm)/CuPc (50 nm)/α-NPB (50 nm)/Compound S:perylene (40:0.3 nm)/Liq (30 nm)/LiF (0.5 nm)/Al

where S is as shown below.

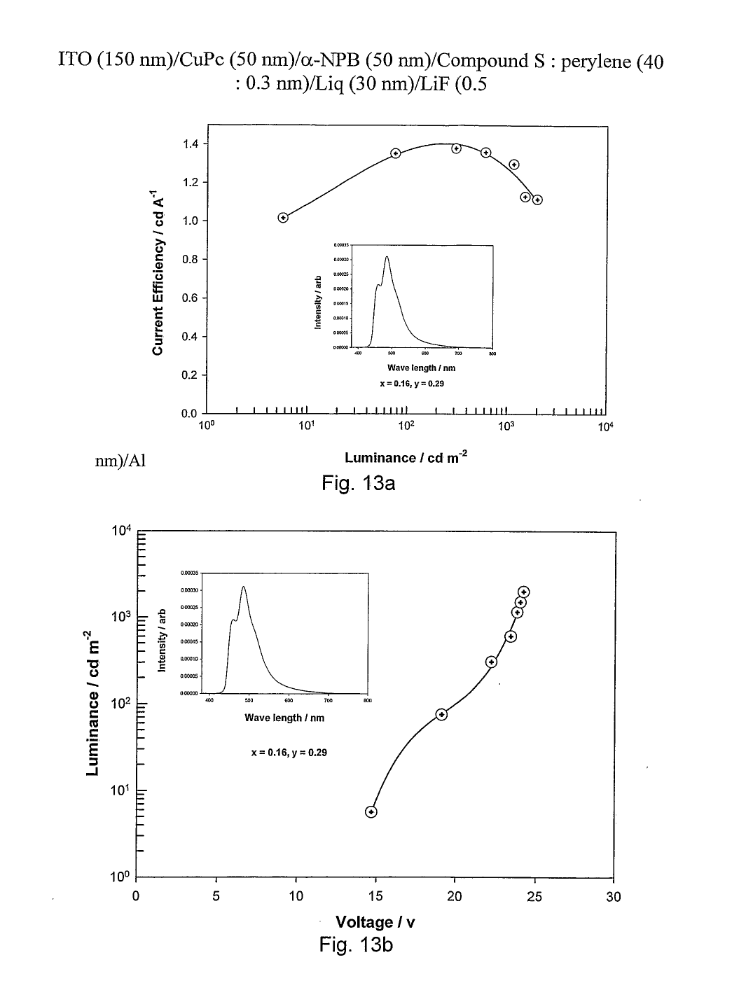

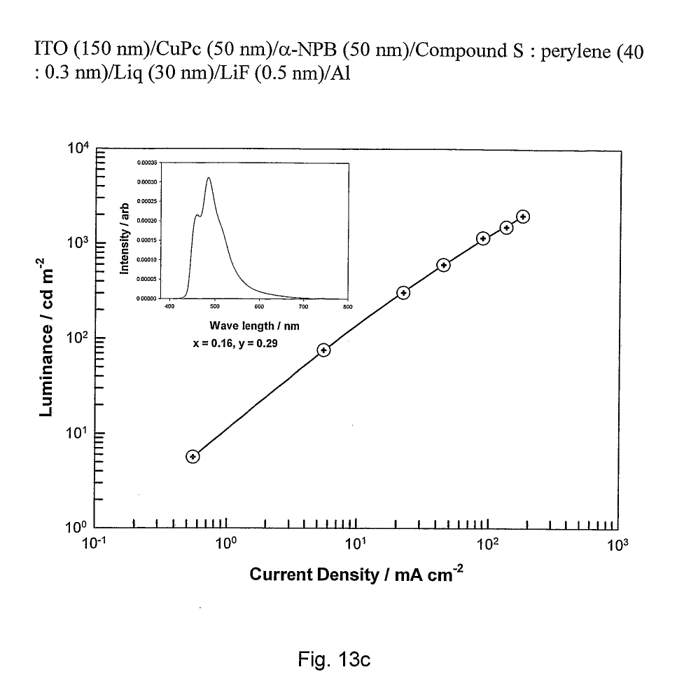

A voltage was applied across the device and the properties measured and the results are shown in FIGS. 13a, 13b and 13c.

### Example 15

A device was formed as in Example 10 with the structure: —

### ITO (110 nm)/ZnTpTP (20 nm)/α-NPB (60 nm)/Compound S (20 nm)/Hfq₄ (30 nm)/LiF (0.3 nm)/Al

where ZnTpTp and S are as shown below.

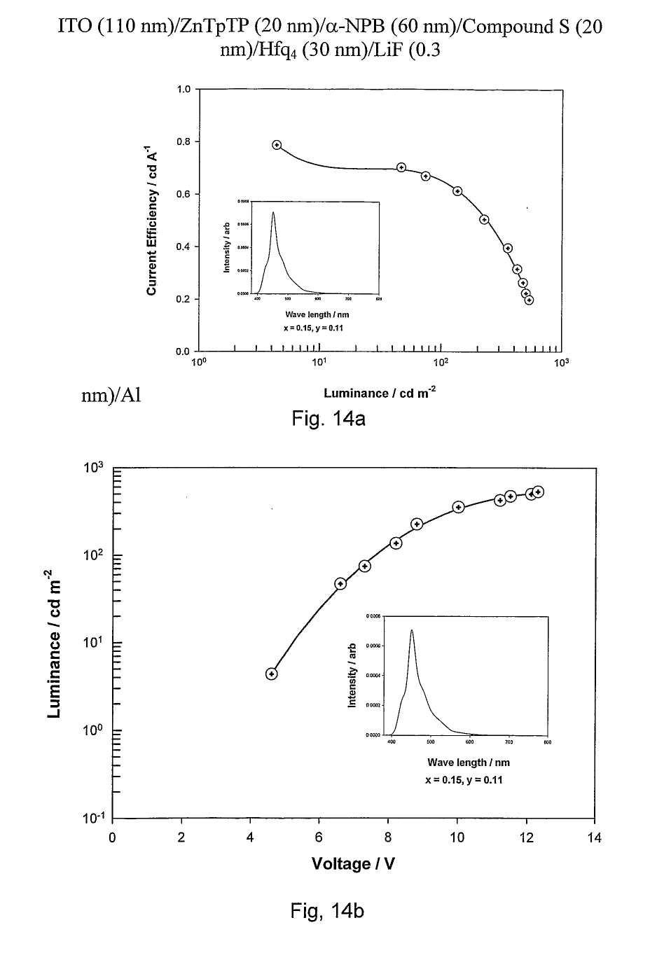

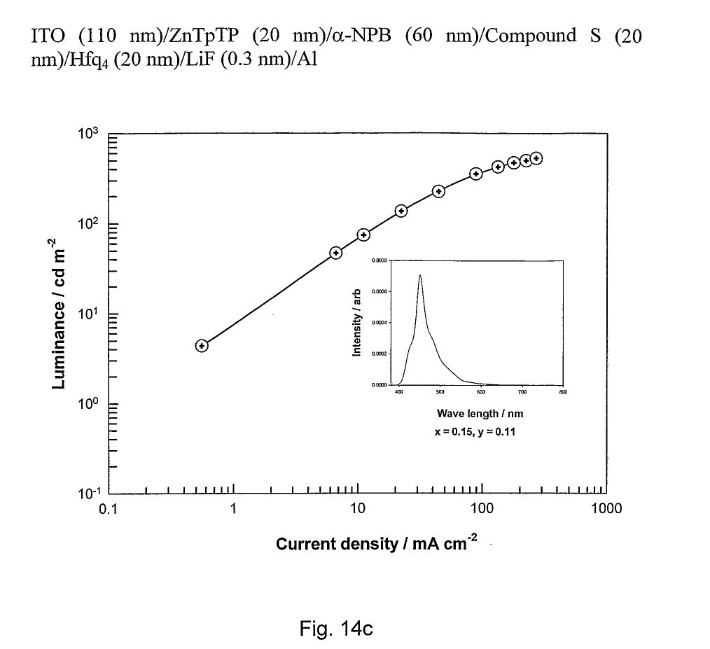

A voltage was applied across the device and the properties measured and the results are shown in FIGS. 14a, 14b and 14c.

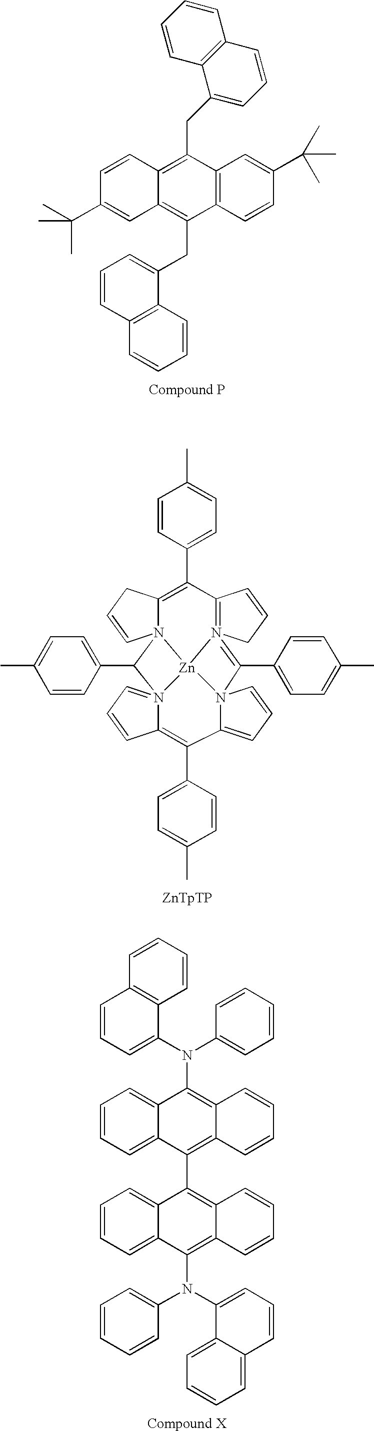

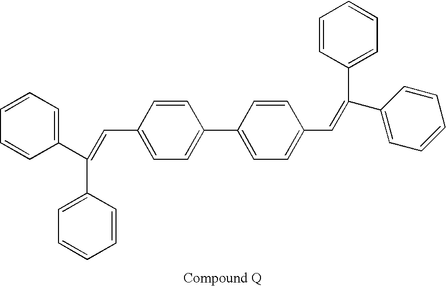

1.-24. (canceled)

25. An electroluminescent compound of formula 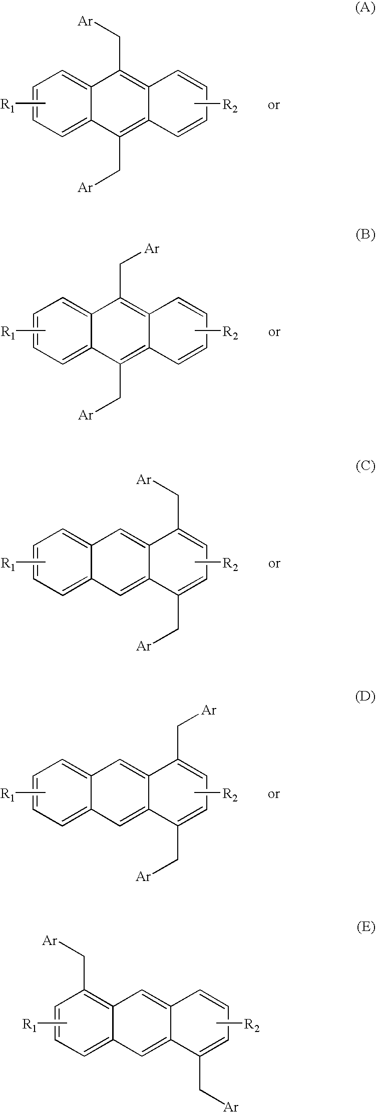
wherein:
Ar is tertiary alkyl or is a substituted or unsubstituted aromatic group; and
R₁ and R₂ may be the same or different and are selected from the group consisting of hydrogen, hydrocarbyl groups, substituted and unsubstituted aliphatic groups, aromatic groups, heterocyclic groups fluorocarbon groups and polycyclic ring structures, or R₁ and R₂ may together form substituted or unsubstituted fused aromatic, heterocyclic and polycyclic ring structures and can be copolymerizable with styrene or with another monomer.

26. The compound of claim 25, wherein Ar is selected from

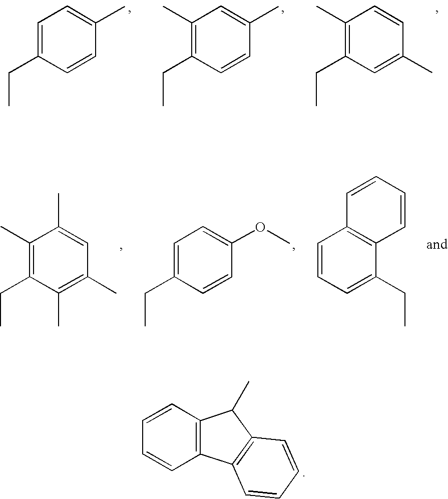

27. Any of the following compounds:
9,10-bis-(4-methylbenzyl)-anthracene;
9,10-bis-(2,4-dimethylbenzyl)-anthracene;
9,10-bis-(2,5-dimethylbenzyl)-anthracene;
9,10-bis-(2,3,5,6-tetramethylbenzyl)-anthracene;
9,10-bis-(4-methoxulbenzyl)-anthracene;
9,10-bis-(9H-fluoren-9-yl)-anthracene;
2.6-di-t-butylanthracene;
2.6-di-t-adamantyl-lanthracene;
2.6-di-t-butyl-9,10-bis-(2,5-dimethylbenzyl)-anthracene;
2.6-di-t-butyl-9,10-bis-naphthalen-1-yl-anthracene.

28. An electroluminescent composition comprising
(i) an electroluminescent compound of formula 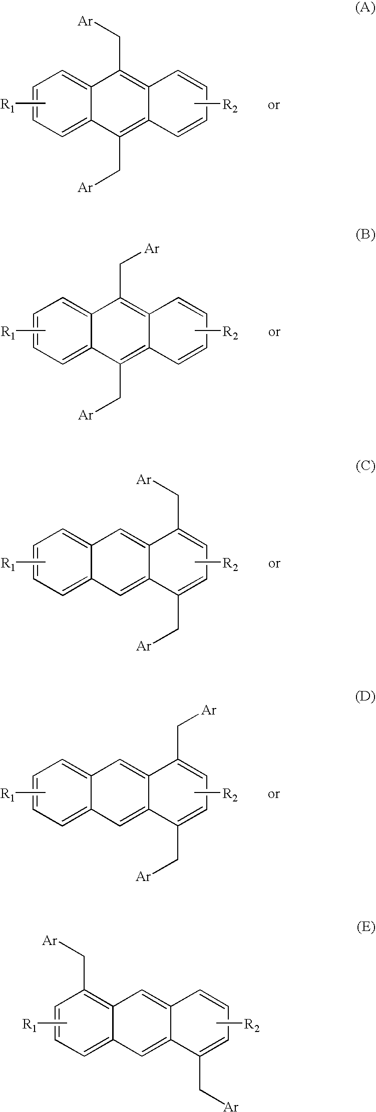
wherein:
Ar is tertiary alkyl or is a substituted or unsubstituted aromatic group; and
R₁ and R₂ may be the same or different and are selected from the group consisting of hydrogen, hydrocarbyl groups, substituted and unsubstituted aliphatic groups, aromatic groups, heterocyclic groups fluorocarbon groups and polycyclic ring structures, or R₁ and R₂ may together form substituted or unsubstituted fused aromatic, heterocyclic and polycyclic ring structures and can be copolymerizable with styrene or with another monomeras defined in claim 1 and
(ii) a host material.

29. The composition of claim 28, wherein the host forms a common phase with the electroluminescent compound.

30. The composition of claim 28, wherein the host is of formula: 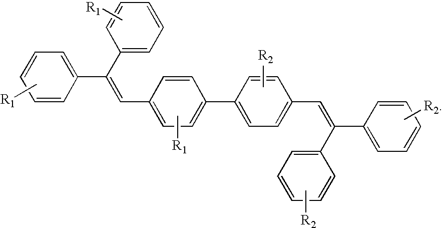
wherein R₁ and R₂ may be hydrogen or substituted or unsubstituted hydrocarbyl.

31. The composition of claim 28, wherein the host is of formula:

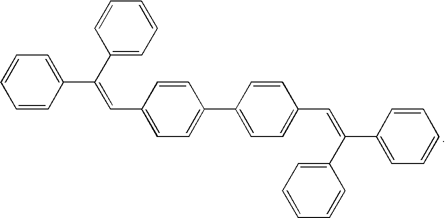

32. An electroluminescent device which comprises
(i) a first electrode,
(ii) a layer comprising an electroluminescent compound of formula 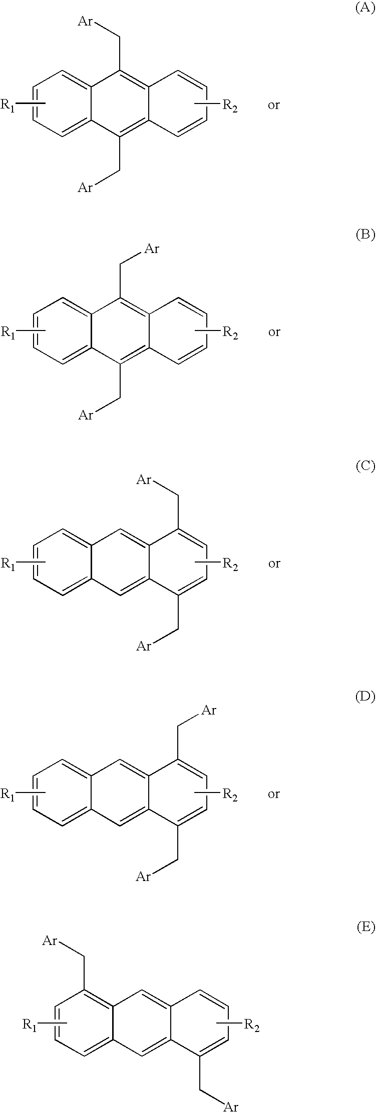
wherein:
Ar is tertiary alkyl or is a substituted or unsubstituted aromatic group; and
R₁ and R₂ may be the same or different and are selected from the group consisting of hydrogen, hydrocarbyl groups, substituted and unsubstituted aliphatic groups, aromatic groups, heterocyclic groups fluorocarbon groups and polycyclic ring structures, or R₁ and R₂ may together form substituted or unsubstituted fused aromatic, heterocyclic and polycyclic ring structures and can be copolymerizable with styrene or with another monomer and
(iii) a second electrode.

33. The device of claim 32, wherein there is a layer of ZnTpTp or of the following compound between the first electrode and the electron injection layer:

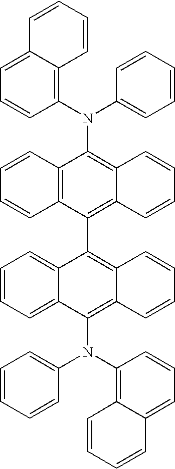

34. The device of claim 32, wherein there is a layer of a hole transmitting material between the first electrode and the electroluminescent layer.

35. The device of claim 34, wherein the hole transmitting material is an aromatic amine compound.

36. The device of claim 34, wherein the hole transmitting layer is of a material selected from:
(a) α-NBP;
(b) a film of a polymer selected from poly(vinylcarbazole), N,N′-diphenyl-N,N′-bis(3-methylphenyl)-1,1′-biphenyl-4,4′-diamine (TPD), polyaniline, substituted polyanilines, polythiophenes, substituted polythiophenes, polysilanes and substituted polysilanes;
(c) a copolymer of aniline, a copolymer of aniline with o-anisidine, m-sulphanilic acid or o-aminophenol, or o-toluidine with o-aminophenol, o-ethylaniline, o-phenylene diamine or with an amino anthracene;
(d) a conjugated polymer selected from poly(p-phenylenevinylene)-PPV and copolymers including PPV, poly(2,5 dialkoxyphenylene vinylene), poly(2-methoxy-5-(2-methoxypentyloxy-1,4-phenylene vinylene), poly(2-methoxypentyloxy)-1,4-phenylenevinylene), poly(2-methoxy-5-(2-dodecyloxy-1,4-phenylenevinylene) and other poly(2,5 dialkoxyphenylenevinylenes) with at least one of the alkoxy groups being a long chain solubilising alkoxy group, poly fluorenes and oligofluorenes, polyphenylenes and oligophenylenes, polyanthracenes and oligo anthracenes, ploythiophenes and oligothiophenes.

37. The device of claim 32, wherein there is a layer of an electron transmitting material between the cathode and the electroluminescent compound layer.

38. The device of claim 37, wherein the electron transmitting material is a metal quinolate or a metal thioxinate.

39. The device of claim 38, wherein the metal quinolate is an aluminium quinolate, zirconium quinolate, hafnium quinolate or lithium quinolate and the metal thioxinate is zinc thioxinate, cadmium thioxinate, gallium thioxinate or indium thioxinate.

40. The device of claim 32, wherein the first electrode is a transparent electricity conducting glass electrode.

41. The device of claim 32, wherein the second electrode is comprised of a metal other than an alkali metal having a work function of less than 4 eV.

42. The device of claim 32, wherein the second electrode is selected from aluminium, calcium, lithium, magnesium and alloys thereof and silver/magnesium alloys.
